(19) United States
(12) Patent Application Publication
(10) Pub. No.: US 2011/0237976 A1
(43) Pub. Date: Sep. 29, 2011

(54) BIOPSY DEVICE HAVING HEMOSTATIC CONTROL

(75) Inventors: William F. Weitzel, Ypsilanti, MI (US); Anne E. Kirkpatrick, Northville, MI (US); David A. Thompson, Fort Gratiot, MI (US); Todd Addis, Lake Ann, MI (US); Christopher M. Welch, Livonia, MI (US); Grant H. Kruger, Ann Arbor, MI (US); Philip M. Wong, Saline, MI (US); Panduranga S. Rao, Ann Arbor, MI (US)

(73) Assignee: The Regents of the University of Michigan, Ann Arbor, MI (US)

(21) Appl. No.: 13/131,482

(22) PCT Filed: Dec. 3, 2009
(86) PCT No.: PCT/US09/66578

§ 371 Date: May 26, 2011

Related U.S. Application Data
(60) Provisional application No. 61/200,787, filed on Dec. 3, 2008.

(51) Int. Cl. A61B 10/02

(52) U.S. Cl. 600/567

(57) ABSTRACT

A biopsy device assembly performs a biopsy of an anatomical tissue of a patient. The biopsy device assembly includes a housing and a biopsy device that extends out of the housing and that collects and cuts anatomical tissue from the patient. The biopsy device assembly further includes a hemostatic agent removably housed in the biopsy device. Moreover, the assembly includes an actuator assembly that moves the biopsy device relative to the housing from a first position to an extended position such that the biopsy device collects and cuts the anatomical tissue from the patient. The actuator assembly also retracts the biopsy device relative to the housing toward a retracted position. Furthermore, the assembly includes an ejection device that ejects the hemostatic agent from the biopsy device as the actuator assembly retracts the biopsy device toward the retracted position.

### CROSS-REFERENCE TO RELATED APPLICATIONS

This application claims the benefit of U.S. Provisional Application No. 61/200,787, filed on Dec. 3, 2008, the entire disclosure of which is incorporated herein by reference.

### GOVERNMENT RIGHTS

This invention was made with government support under DK062848 awarded by the National Institutes of Health. The government has certain rights in the invention.

### FIELD

The present disclosure relates to a biopsy device and, more particularly, to a biopsy device having hemostatic control.

### BACKGROUND

This section provides background information related to the present disclosure which is not necessarily prior art.

Bleeding from biopsy is a major problem in many areas of medicine but particularly in nephrology where the risk of serious bleeding from the kidney, although infrequent, may become life-threatening. While methods of performing kidney biopsies have improved over the last two decades renal biopsies still entail inherent bleeding risk such that fully a third of patients have post biopsy hematoma. Even though most of these hematomas don't become life threatening, the current strategy of monitoring for and managing complications is woefully inadequate. The current standard of practice is to study risk factors associated with bleeding such as hypertension and amyloidosis, mitigating some modifiable risk factors and choosing not to biopsy most patients at increased risk. This is far from optimal medical practice since the risk of bleeding is greater in many patient populations where renal biopsy would be most helpful, such as in autoimmune diseases and renal dysfunction manifested by elevated creatinine to >2 mg/dl. In addition, even mild coagulopathies increase the risk to the point where conventional (percutaneous) renal biopsy is significantly risky. While the published data tend to indicate that the serious bleeding complications may occur only 1 to 2% of the time, these data are inherently biased by physician practice patterns excluding the riskiest patients from percutaneous renal biopsy. This approach has led to the current practice of close clinical observation, with escalating anxiety, especially when transfusions become necessary when the patient's hematocrit is falling. In this setting, the next step is to proceed with invasive and risky treatments to address the excessive bleeding by performing renal arteriography and segmental embolization or surgery.

In order to respond to the bleeding risk, physicians appropriately limit percutaneous renal biopsy to cases where the diagnostic information exceeds the potentially life threatening risk to the patient. In settings where conventional biopsy is considered too risky, high risk patients needing kidney biopsy are referred to interventional radiologists and surgeons to perform more complicated invasive procedures such as open (surgical) biopsy or transjugular renal biopsy. The open procedure has considerably more morbidity and cost, still entails bleeding risk, and the transjugular procedure is much more invasive than conventional biopsy and merely serves to keep the bleeding that does occur within the vascular space (i.e., bleeding is directed into the venous system). Advances in laparoscopic procedures have allowed a less morbid surgical approach to be used, but this remains considerably more involved and costly than the percutaneous approach and is reserved for cases where the standard percutaneous approach is contra-indicated.

The alternative transjugular procedure entails inserting a wire, followed by a biopsy device into the neck (jugular vein) and navigating the device using fluoroscopic (video X-ray) guidance through the veins in the chest, right atrium of the heart, into the inferior vena cava, and finally into the renal vein, inserting the device up though the central regions of the kidney, where the needle can be pushed through the interior portions of the kidney, eventually making its way up into the outer regions of the kidney cortex, where the diagnostically useful region of the kidney is located. The reason this more invasive, less desirable approach is used is because the bleeding that does occur is bleeding within the venous system that remains in the circulation rather than outside of the kidney. This procedure requires costly equipment and a highly skilled operator, but if performed correctly, then bleeding risk is reduced. However, systematic study at leading institutions shows this procedure causes intraperitoneal bleeding just as does the standard percutaneous biopsy. The transjugular procedure has a yield (samples containing renal tissue with glomeruli) of only about 80%. Therefore, while the transjugular approach has allowed renal biopsies to be obtained with greater safety, there is still need to improve the safety of this procedure and the added complexity clearly keep it from being an acceptable alternative to percutaneous biopsy.

### SUMMARY

This section provides a general summary of the disclosure, and is not a comprehensive disclosure of its full scope or all of its features.

A biopsy device assembly that performs a biopsy of an anatomical tissue of a patient is disclosed. The biopsy device assembly includes a housing and a biopsy device that extends out of the housing and that collects and cuts anatomical tissue from the patient. The biopsy device assembly further includes a hemostatic agent removably housed in the biopsy device. Moreover, the assembly includes an actuator assembly that moves the biopsy device relative to the housing from a first position to an extended position such that the biopsy device collects and cuts the anatomical tissue from the patient. The actuator assembly also retracts the biopsy device relative to the housing toward a retracted position. Furthermore, the assembly includes an ejection device that ejects the hemostatic agent from the biopsy device as the actuator assembly retracts the biopsy device toward the retracted position.

Moreover, a method of performing a biopsy is disclosed. The method includes locating a biopsy device relative to a target location within a patient. The biopsy device extends from a housing. The method also includes actuating the biopsy device relative to the housing from a first position to an extended position such that the biopsy device collects and cuts an anatomical tissue from the patient. Furthermore, the method includes retracting the biopsy device relative to the housing toward a retracted position. Still further, the method includes ejecting a hemostatic agent from the biopsy device as the biopsy device is retracted toward the retracted position.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a perspective view of various exemplary embodiments of a biopsy device according to teachings of the present disclosure;

FIG. 2 is an exploded perspective view of the biopsy device of FIG. 1;

FIG. 3 is a partially exploded view of the biopsy device of FIG. 1;

FIG. 4 is a section view of the biopsy device of FIG. 1 shown in a first stage of deployment;

FIG. 5 is a section view of the biopsy device of FIG. 1 shown in a second stage of deployment;

FIG. 6 is a section view of the biopsy device of FIG. 1 shown in a third stage of deployment;

FIG. 7 is a perspective view of a portion of the biopsy device of FIG. 5 shown in the second stage of deployment;

FIG. 8 is a perspective view of a portion of the biopsy device of FIG. 6 shown in the third stage of deployment;

FIG. 9 is a perspective view of a hemostatic plug according to various exemplary embodiments of the present disclosure;

FIG. 10 is a perspective view of a distal end of the biopsy device;

FIG. 11 is a section view of another exemplary embodiment of the biopsy device of the present disclosure;

FIG. 12 is a section view of the biopsy device of FIG. 11 in a first stage of deployment;

FIG. 13 is a section view of the biopsy device of FIG. 11 in a second stage of deployment; and

FIG. 14 is a section view of the biopsy device of FIG. 11 in a third stage of deployment.

Corresponding reference numerals indicate corresponding parts throughout the several views of the drawings.

### DETAILED DESCRIPTION

Example embodiments will now be described more fully with reference to the accompanying drawings.

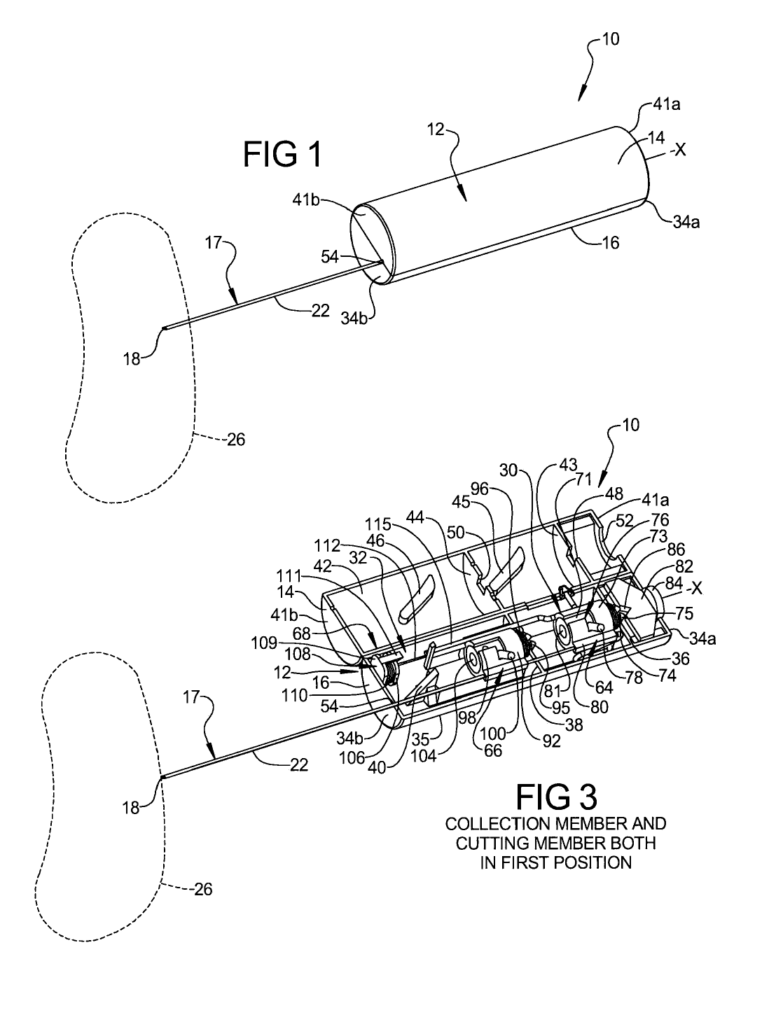

Referring initially to FIGS. 1-8, a biopsy device assembly 10 is illustrated according to various exemplary embodiments of the present disclosure. As will be discussed, the biopsy device assembly 10 can perform a biopsy of tissue from an anatomical feature 26 (FIG. 1) of a patient. For instance, the anatomical feature 26 can be a kidney, and the biopsy device assembly 10 can be used to perform a biopsy of kidney tissue. However, it will be appreciated that the biopsy device assembly 10 can be configured for performing a biopsy of any suitable anatomical feature 26 without departing from the scope of the present disclosure.

The biopsy device assembly 10 can generally include a housing 12 as shown in FIGS. 1-3. The housing 12 can include a first shell member 14 and a second shell member 16 that are joined together to define a cavity within the housing 12. The housing 12 will be described in greater detail below.

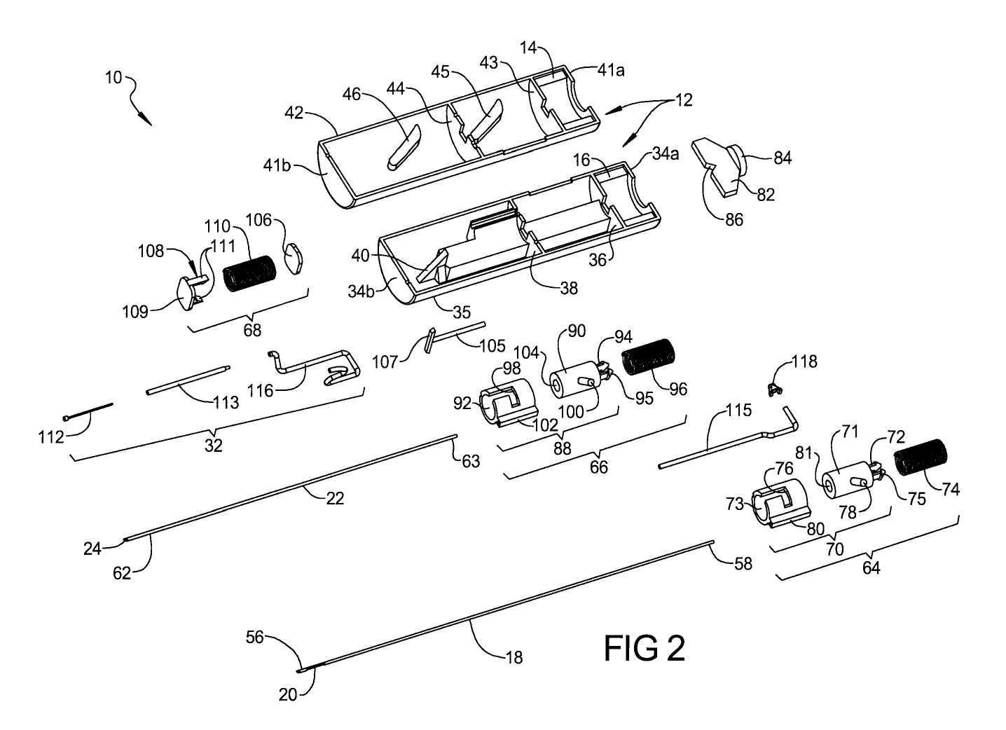

The biopsy device assembly 10 can also include a biopsy device, generally indicated at 17 in FIGS. 1 and 2. The biopsy device 17 can extend out of the housing 12 and can collect and cut anatomical tissue from the anatomical feature 26, as will be discussed.

As shown in FIG. 10, the biopsy device 17 can include a collection member 18, such as an elongate needle. The collection member 18 can include a collection recess 20 (shown in phantom) on an outer surface of the collection member 18 as will be discussed in greater detail below. The collection member 18 can also include a cannula 21, which will be described in greater detail below. The biopsy device 17 can also include a cutting member 22, such as a hollow tube. The cutting member 22 can include a cannula 24 that moveably receives the collection member 18, as shown in FIG. 10.

It will be appreciated that the biopsy device 17 can be of any suitable type for collecting, cutting, and/or retaining the tissue of the anatomical feature 26 to perform the biopsy. As such, the biopsy device 17 may or may not include both the collection member 18 and the cutting member 22.

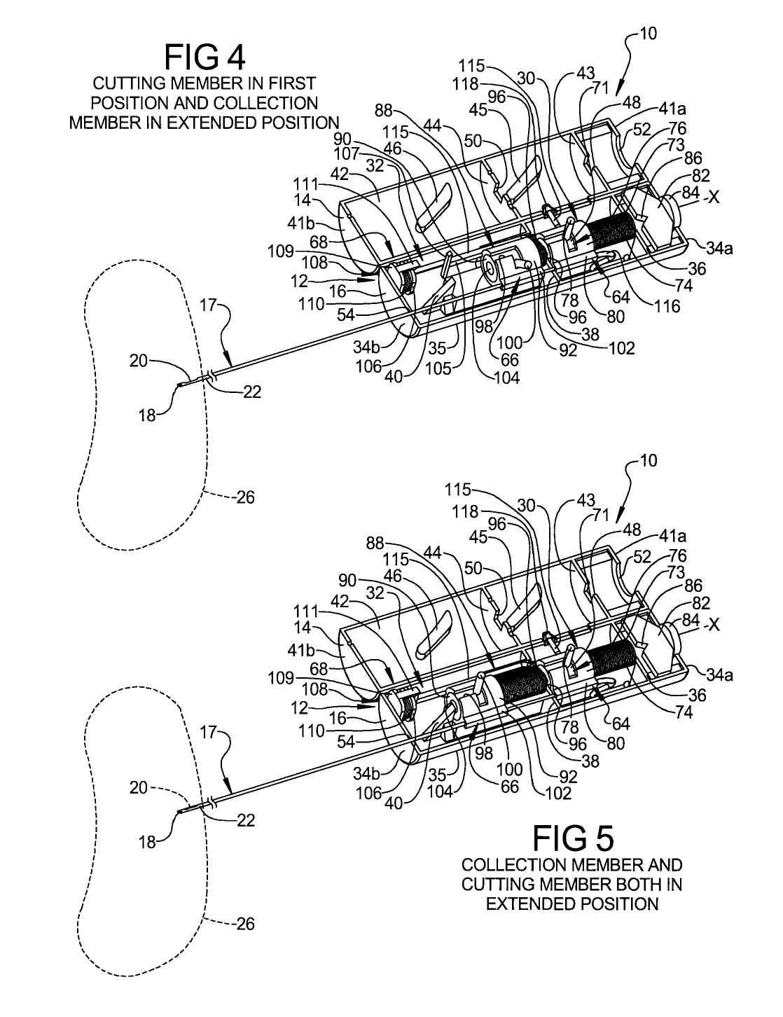

In addition, the biopsy device assembly 10 can include an actuator assembly, which is generally indicated at 30 in FIGS. 3-8. As will be discussed, the actuator assembly 30 can actuate and independently move the collection member 18 and the cutting member 22 of the biopsy device 17. For instance, the actuator assembly 30 can independently actuate the collection member 18 and the cutting member 22 along an axis X of the assembly 10 relative to the housing 12. Specifically, the actuator assembly 30 can move the collection member 18 and the cutting member 22 from a first position (locked and loaded position) (FIG. 3) to an extended position (FIGS. 4 and 5) into the anatomical feature 26 in order to perform the biopsy procedure.

More specifically, the actuator assembly 30 can move the collection member 18 from a respective first position (FIG. 3) to a respective extended position (FIG. 4) to extend out of the cannula 24 of the cutting member 22 (e.g., approximately 22 millimeters) such that the collection recess 20 is exposed and receives anatomical tissue of the anatomical feature 26. (The natural elasticity of the anatomical feature 26 can cause tissue to move into the collection recess 20.) Then, the actuator assembly 30 can move the cutting member 22 from a respective first position (FIG. 4) to a respective extended position (FIG. 5) to thereby cut tissue disposed in the collection recess 20 away from the anatomical feature 26 and to cover and secure the tissue within the collection recess 20.

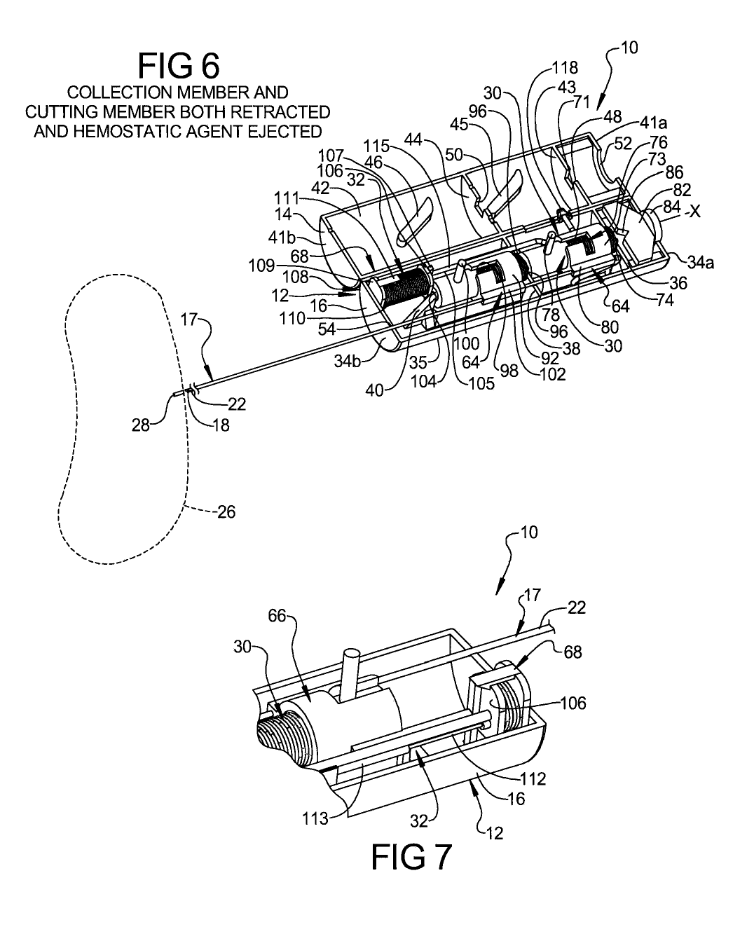

Furthermore, as will be discussed, the actuator assembly 30 can retract the collection member 18 and the cutting member 22 from the respective extended positions (FIG. 5) to respective retracted positions (FIG. 6) to move away from the anatomical feature 26. Subsequently, a medical professional can retrieve the anatomical tissue from the collection recess 20 to perform suitable analytical tests on the tissue.

Moreover, the biopsy device assembly 10 can further include a hemostatic agent 28, best illustrated in FIG. 10. The hemostatic agent 28 can be of any suitable type for reducing bleeding caused by the biopsy procedure. The hemostatic agent 28 can be removeably housed within the cannula 21 of the collection member 18.

Additionally, the biopsy device assembly 10 can include an ejection device 32, which is generally indicated at 32 in FIGS. 2-8. The ejection device 32 can eject the hemostatic agent 28 from the cannula 21 of the collection member 18 as the actuator assembly 30 retracts the collection member 18 and cutting member 22 from the respective extended positions (FIG. 5) toward the respective retracted positions (FIG. 6). The ejection device 32 can eject the hemostatic agent 28 into the anatomical feature 26 as the collection member 18 and cutting member 22 are retracted (i.e., the hemostatic agent 28 can be ejected into the void in the anatomical feature 26 created by the collection member 18 and the cutting member 22).

Thus, because the hemostatic agent 28 is housed substantially completely within the cannula 21 of the collection member 32 during extension of the collection member 18 and cutting member 22 into the anatomical feature 26, the hemostatic agent 28 can remain protected until it is ready to be ejected. Then, the hemostatic agent 28 can be automatically ejected into the void as the collection member 18 and the cutting member 22 are retracted, such that the hemostatic agent 28 is likely to be placed accurately and effectively to reduce bleeding.

Referring now to FIGS. 2 and 3, the housing 12 will be described in greater detail. As shown, the housing 12 can be generally hollow and cylindrical and can include the first shell member 14 and the second shell member 16. The first and second shell members 14, 16 can be made out of any suitable material, such as DELRIN plastic.

The first member 14 can include two semi-circular ends 34a, 34b and an outer curved wall 35 that extends between the ends 34a, 34b. Furthermore, the first member 14 can include a first substantially semi-circular wall 36 and a second substantially semi-circular wall 38 that are spaced apart from each other and that are spaced apart axially from the ends 34a, 34b. In addition, the first member 14 can include a triangular stop 40 that extends radially from an inner surface of the outer wall 35 between the second wall 38 and the end 34b.

The second member 16 of the housing 12 can be substantially similar to the first member 14 and can include ends 41a, 41b and an outer wall 42. Moreover, the second members 16 can include a first wall 43 and a second wall 44. In addition, as shown in FIGS. 2 and 3, the second member 16 can include a first cam member 45 and a second cam member 46. The first and second cam members 45, 46 can protrude from the inner surface of the outer wall 42 and can extend helically about the axis X. The first cam member 45 can be disposed between the first walls 36, 43 and the second walls 38, 44, and the second cam member 46 can be disposed between the second walls 38, 44 and the ends 34b, 41b. The first and second cam members 45, 46 can cam portions of the actuator assembly 30, as will be discussed in greater detail below.

When the first and second members 14, 16 of the housing 12 are joined (FIG. 1), the ends 34a, 34b can be fixed to the ends 41a, 41b, respectively. Also, the first wall 36 of the first member 14 can join to the first wall 43 of the second member 16, and the second wall 38 of the first member 14 can join to the second wall 44 of the second member 16.

Moreover, when the first and second members 14, 16 of the housing 12 are joined, the housing 12 can include a first end opening 52, a second end opening 54, a first central opening 48, and a second central opening 50. (The second end opening 54 is shown in FIG. 1, but since FIG. 3 shows the housing 12 exploded, the first end opening 52, the first central opening 48, and the second central opening 50 are indicated on the first member 14 only.) The end 34a of the first member 14 and the end 41a of the second member 16 can cooperate to define the first end opening 52. Likewise, the end 34b of the first member 14 and the end 41b of the second member 16 can cooperate to define the second end opening 54. Moreover, the first walls 36, 43 can cooperate to define the first central opening 48. Likewise, the second walls 38, 44 can cooperate to define the second central opening 50. The first end opening 52 and the first and second central openings 48, 50 can be centered on the axis X, and the second end opening 54 can be spaced radially away from the axis X.

Referring now to FIG. 2, the collection member 18 will be described in greater detail. As shown, the collection member 18 can be an axially straight needle. The collection member 18 can be made out of any suitable material, such as metal (e.g., stainless steel). The collection member 18 can include a distal end 56, which can be sharpened. For instance, the distal end 56 can be cut at an angle relative to the axis X, such that the distal end 56 is sharp enough to pierce and penetrate the anatomical feature 26. In addition, the collection member 18 can include a proximal end 58.

As shown in FIG. 10, the collection recess 20 of the collection member 18 can extend depth-wise, radially inward and can extend length-wise longitudinally along the axis X. The collection recess 20 can be disposed adjacent the distal end 56. Moreover, as shown in FIG. 10, the cannula 21 can extend from the distal end 56 substantially parallel to the axis X, and the cannula 21 can extend continuously from the distal end 56 (i.e., the first terminal end) to the proximal end 58 (i.e., the second terminal end) of the collection member 18.

Referring back to FIG. 2, the cutting member 22 will be described in greater detail. The cutting member 22 can be a hollow tube that is axially straight. As such, the collection member 18 can include a distal end 62 and a proximal end 63. The cutting member 22 can be made out of any suitable material, such as stainless steel. Moreover, the cutting member 22 can include the cannula 24, which extends continuously from the distal end 62 (i.e., the first terminal end) to the proximal end 63 (i.e., the second terminal end). The cannula 24 can extend substantially parallel to the axis X.

Moreover, referring to FIGS. 2 and 3, the actuator assembly 30 will be discussed in greater detail. The actuator assembly 30 can generally include a first portion 64, and second portion 66, and a third portion 68. As will be discussed, the first portion 64 can actuate the collection member 18, the second portion 66 can actuate the cutting member 22, and the third portion 68 can actuate each of the collection member 18, the cutting member 22, and the ejection device 32.

As shown in FIGS. 2 and 3, the first portion 64 of the actuator assembly 30 can include a first stage 70, a first retainer 72, and a first biasing member 74. The first stage 70 can include an inner cylinder 71 and an outer cylinder 73, and the first retainer 72 can be fixed to the inner cylinder 71.

The inner cylinder 71 can be substantially cylindrical and can be made out of any suitable material, such as plastic (e.g., DELRIN plastic). The inner cylinder 71 can include a conical recess 81 on one end, and the recess 81 can be centered on the axis X. The first retainer 72 can extend axially from the opposite end of the inner cylinder 71. In some embodiments, there are a plurality (e.g., two) of resilient, spaced apart first retainers 72 with enlarged retaining heads 75. The inner cylinder can also include a protrusion 78, such as a peg that extends transverse (e.g., perpendicular) to the axis X.

Moreover, the outer cylinder 73 can be substantially hollow and cylindrical and can moveably receive the inner cylinder 71 therein. Furthermore, the outer cylinder 73 can include a track 76. The track 76 can be generally L-shaped to include a portion that extends circumferentially about the outer cylinder 73 and a portion that extends parallel to the axis X. As will be discussed, the protrusion 78 can be moveably received within the track 76 of the outer cylinder 73. Furthermore, the outer cylinder 73 can include a holder 80, which extends outward from the axis of the outer cylinder 73. The holder 80 can receive and fixably retain the proximal end 58 of the collection member 18.

Additionally, the first biasing member 74 can be of any suitable type and can be made of any suitable material. In some embodiments, the first biasing member 74 is a helical compression spring made out of stainless steel. The first biasing member 74 can be wound about the inner cylinder 71.

As shown in FIG. 3, when the first portion 64 of the actuator assembly 30 is assembled within the housing 12, the inner and outer cylinders 71, 73 and the first biasing member 74 can be disposed between the first walls 36, 43 and the second walls 38, 44 of the housing 12. The first retainer 72 can extend through the first central opening 48 such that the retaining heads 75 are retained against and releasably engaged with the first walls 36, 43. Also, the protrusion 78 of the inner cylinder 71 can be positioned within the track 76 such that the inner cylinder 71 releasably engages the outer cylinder 73. When the first portion 64 is retained as shown in FIG. 3, the first biasing member 74 can be compressed between the inner cylinder 71 and the first walls 36, 43.

Thus, the first portion 64 of the actuator assembly 30 can have a first position (i.e., locked, spring-loaded position), which is represented in FIG. 3. Since the collection member 18 is connected to the outer cylinder 73, the first position of the first portion 64 can correspond to the first position 64 of the collection member 18.

As shown in FIGS. 2 and 3, the second portion 66 of the actuator assembly 30 can be substantially similar to the first portion 64. More specifically, the second portion 66 can include a second stage 88 with an inner and outer cylinder 90, 92, a plurality of second retainers 94 with retainer heads 95, and a second biasing member 96. Also similar to the first portion 64, the inner cylinder 90 can include a protrusion 100 which is moveably received in a track 98 of the outer cylinder 92. Furthermore, the outer cylinder 92 can include a holder 102 that receives and fixably retains the proximal end 63 of the cutting member 22.

When the second portion 66 is in its respective first position (i.e., locked, spring-loaded position) represented in FIG. 3, the second retainers 94 can extend through the second central opening 50 such that the retainer heads 95 releasably engage the second walls 38, 44. Moreover, the second biasing member 96 can be compressed between the second walls 38, 44 and the inner cylinder 90. Since the cutting member 22 is fixed to the outer cylinder 92, the first position of the cutting member 22 can correspond to the first position of the second portion 66 of the actuator assembly 30.

Referring now to FIGS. 2 and 3, the third portion 68 of the actuator assembly 30 will now be described. As shown, the third portion 68 can include a third stage 106, a third retainer 108, and a third biasing member 110. The third stage 106 and third retainer 108 can be made out of any suitable material, such as DELRIN plastic, and the third biasing member 110 can be made out of any suitable material, such as stainless steel. The third stage 106 can be a flat, rectangular plate. The third retainer 108 can include a flat plate 109 and one or more integrally connected clips 111. The plate 109 can be fixed to the ends 34b, 41b of the housing 12, and the clips 111 can extend substantially parallel to the axis X. The third biasing member 110 can be a helical compression spring (e.g., a stainless steel spring). The third biasing member 110 can be disposed between the plate 109 and the third stage 106.

The third portion 68 can have a respective first position (i.e., locked, spring-loaded position) represented in FIG. 3. In this position, the clips 111 can releasably engage the stage 106, and the third biasing member 110 can be compressed between the stage 106 and the plate 109.

As shown in FIGS. 2 and 3, the first portion 64 of the actuator assembly 30 can also include an arm 115. The arm 115 can be a rod that is fixed at one end to the outer cylinder 73, on a side of the axis X opposite from the holder 80. The other end of the arm 115 can extend freely toward the stage 106 of the third portion 68 of the actuator assembly 30.

Moreover, as shown in FIGS. 2 and 3, the second portion 66 of the actuator assembly 30 can also include an arm 105 with an abutment member 107. The arm 105 can be a rod that is fixed at one end to the outer cylinder 92. The abutment member 107 can be fixed to an opposite end of the arm 105, generally adjacent the stage 106 of the third portion 68 of the actuator assembly 30.

As shown in FIGS. 2 and 3, the biopsy device assembly 10 can also include a control 82. The control 82 can be relatively flat and plate-like, and as shown in FIG. 3, the control 82 can be disposed between the ends 34a, 41a and the first walls 36, 43 of the housing 12. The control 82 can be slidingly disposed within the housing 12 so as to slide generally parallel to the axis X of the assembly 10. The control 82 can also include a button 84, which is moveably received within the first end opening 52. The control 82 can also include a substantially triangular recess 86, which is disposed opposite the button 84 and centered on the axis X.

Referring now to FIGS. 2, 3, 7, and 8, the ejection device 32 will be discussed in greater detail. The ejection device 32 can include a plunger 112 (FIGS. 2, 3, 7, 8) and a tube 113 (FIGS. 2, 7, and 8). The tube 113 can be hollow so as to define a cavity therein. The plunger 112 can be received within the cavity of the tube 113. More specifically, the plunger 112 can be slidably received within the tube 113 and can substantially seal to the inner surface of the tube 113. Thus, the plunger 112 and tube 113 can function substantially similar to a syringe. The plunger 112 can also be fixed at one end to the third stage 106. The tube 113 can be fixed to the second member 16 of the housing 12. Also, the cavity in the tube 113 can be filled with an incompressible fluid, such as saline.

Moreover, the ejection device 32 can include a length of tubing 116. In some embodiments, the tubing 116 can be flexible. The tubing 116 can be in fluid communication at one end to the tube 113 and can be in fluid communication with the proximal end 58 of the cannula 21 of the collection member 18.

Thus, as will be discussed, movement of the third stage 106 can cause the plunger 112 to advance into the cavity of the tube 113 to increase pressure in the cavity of the tube 113. As a result, pressure can increase in the cannula 21 of the collection member 18, thereby causing the hemostatic agent 28 to be pushed out of the cannula 21 and into the anatomical feature 26.

The hemostatic agent 28 can be of any suitable type. For instance, as shown in FIG. 9, the hemostatic agent 28 can include a hemostatic foam 120, such as GELFOAM, which is commercially available from Pfizer, Inc. of New York. As such, the hemostatic foam 120 can be water-insoluble and can be absorbent. Also, the hemostatic foam 120 can be made from purified porcine skin gelatin that expands in size as it absorbs blood.

The hemostatic agent 28 can also include a stiffener 122. The stiffener 122 can include an elongate backbone 124 and a plurality of ribs 126. The ribs 126 can be spaced apart from each other and can each be coupled to the elongate backbone 124. Furthermore, as shown in FIG. 9, the foam 120 can be disposed between the ribs 126. The stiffener 122 can be made out of any suitable bio-compatible material, such as polylactic glycolic acid. Moreover, the stiffener 122 can be formed in any suitable fashion, such as injection molding. Also, the foam 120 can molded around the stiffener 122. Thus, the hemostatic agent 28 can have an elongate shape. The hemostatic agent 28 can have any suitable width, such as approximately 0.008″, and can have any suitable length, such as approximately 22 mm.

It will be appreciated that the stiffener 122 can reinforce the hemostatic agent 28 to withstand the pressure of deployment from the cannula 21 and to advance against any friction into the anatomical feature 26. Also, the hemostatic foam 120 can substantially reduce bleeding of the anatomical feature 26. It will be appreciated that both the foam 120 and the stiffener 122 can reduce (e.g., resorb) within the anatomical feature 26 after the anatomical feature 26 has healed.

In addition, the biopsy device assembly 10 can include a handle member 118 (FIGS. 2, 4, and 6). The handle member 118 can be fixed to the second arm 115 and can extend out of the housing 112. With the handle member 118, the user can push the second arm 115 along the axis X to push the outer cylinder 73 of the first stage 70 toward the ends 34b, 41b of the housing 12. As such, the collection member 18 can extend out of the cutting member 22 to allow the user to remove the anatomical tissue from the collection recess 20 for further processing.

Referring now to FIGS. 3 through 8, operation of the assembly 10 will be discussed in greater detail. With the collection member 18 and the cutting member 22 in the first position shown in FIG. 3, the user can pierce the patient's skin and guide the distal ends 56, 62 of the collection member 18 and cutting member 22 toward a target location of the anatomical feature 26. The user can rely on imaging systems, such as ultrasound for additional guidance toward the target location.

Then, the user can depress the button 84 to move the control 82 along the axis X. Eventually, the retaining heads 75 of the first retainers 72 will be received within the recess 86 of the control 82. Further axial movement of control 82 can cause the first retainers 72 to resiliently move toward the axis X, eventually causing the retaining heads 75 to release the walls 36, 43 and to move through the first central opening 48. As such, the first biasing member 74 can push against the first walls 36, 43 of the housing 12 and the inner cylinder 71, thereby biasing the inner cylinder 71, the outer cylinder 73, and the collection member 18 toward the extended position shown in FIG. 4. As such, tissue can be received in the collection recess 20 of the collection member 18. Also, this movement causes the arm 115 to move toward and immediately adjacent the third stage 106 (FIG. 4).

It will be noted that as the inner cylinder 71 is biased to the extended position, the first cam member 45 of the housing 12 can cam against the protrusion 78 of the inner cylinder to camingly rotate the inner cylinder 71 about the axis X relative to the outer cylinder 73. As such, the protrusion 78 can move within the track 76 until the protrusion 78 reaches the portion of the track 76 that is parallel to the axis X. As such, this camming motion can cause the outer cylinder 73 to become disengaged from the inner cylinder 71 for purposes that will discussed in greater detail below.

Axial movement of the inner cylinder 71 also subsequently causes the recess 81 of the inner cylinder 71 to receive the second retaining heads 95 of the second portion 66 of the actuator assembly 30. Thus, similar to the first portion 64, the second retaining heads 95 release from the second walls 38, 44, and the second biasing member 96 biases the inner and outer cylinders 90, 92 toward the stop 40 (FIG. 5). This consequently causes cutting member 22 to move to the extended position to cut the anatomical tissue and retain the tissue within the collection recess 20. Moreover, the protrusion 100 cams against the cam member 46 to disengage the outer cylinder 92 from the inner cylinder 90.

In addition, this also causes the abutment member 107 to move toward the third retainer 108 (FIG. 6). As a result, the abutment member 107 cams the clip 111 away from the axis X to release the third stage 106.

Once the third stage 106 is released, the third biasing member 110 biases the third stage 106 away from the plate 109. As a result, the third stage 106 abuts and pushes both the abutment member 107 and the arm 115 along the axis X away from the plate 109. Because both outer cylinders 73, 92 are disengaged from the respective inner cylinders 71, 90, the outer cylinders 73, 92 slide along the axis X and over the first and second biasing members 74, 96. This causes both the collection member 18 and the cutting member 22 to simultaneously move from the extended position (FIG. 5) to the retracted position (FIG. 6).

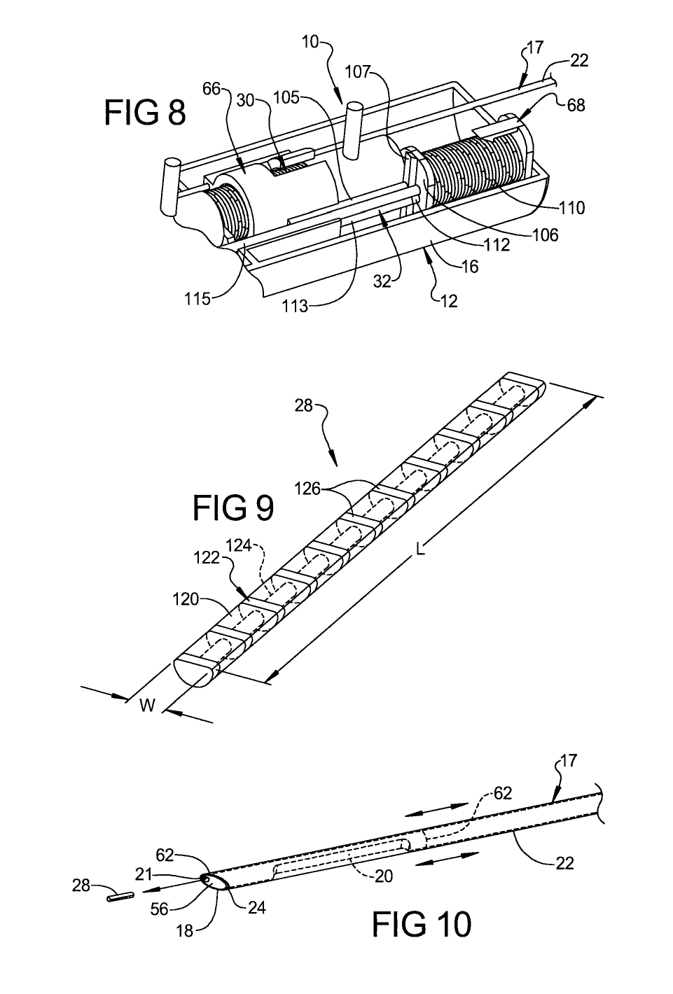

Moreover, as shown in FIGS. 7 and 8, movement of the third stage 106 away from the plate 109 advances the plunger 112 into the tube 113, thereby increasing pressure in the tube 113 and the cannula 21. As a result, the hemostatic agent 28 is ejected from the cannula 21 and into the anatomical feature 26 as the collection member 18 and cutting member 22 are retracted.

It will be appreciated that “ejection” of the hemostatic agent 28 can include a positive force and/or pressure being applied to the hemostatic agent 28 to move the hemostatic agent 28 out of the cannula 21 and such that the hemostatic agent 28 moves relative to the anatomical feature 26 during ejection. It will also be appreciated that “ejection” of the hemostatic agent 28 can include the collection member 18 merely withdrawing from the anatomical feature 26 and leaving the hemostatic agent 28 in a fixed position relative to the anatomical feature 26.

Next, the user can remove the assembly 10 from the patient. The user can then use the handle member 118 as discussed above to extend the collection member 18 out of the cutting member 22 in order to remove the tissue sample located in the collection recess 20.

Accordingly, the assembly 10 allows biopsy procedures to be performed conveniently and accurately. In addition, the hemostatic agent 28 can be shielded within the collection member 18 as the biopsy is collected and cut from the patient. Furthermore, the hemostatic agent 28 can be automatically inserted into the void created by the collection member 18 and cutting member 22 after the collection member 18 and cutting member 22 are retracted and withdrawn from the anatomical feature 26. Accordingly, the hemostatic agent 28 can be conveniently and accurately positioned within the anatomical feature 26, and excessive bleeding is unlikely to occur.

Referring now to FIGS. 11-14, another exemplary embodiment of the biopsy device assembly 210 is illustrated according to various other teachings of the present disclosure. Components that correspond to those above in the embodiments of FIGS. 1-10 are indicated by corresponding reference numerals, increased by 200.

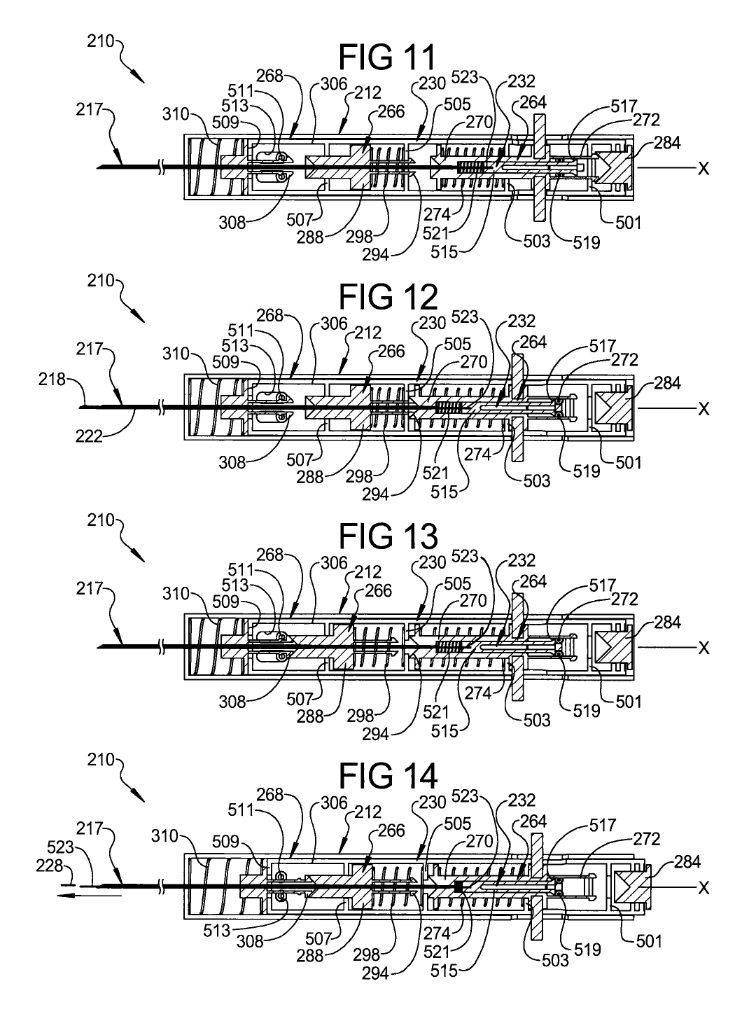

As shown in FIG. 11, the assembly 210 can include a housing 212, a biopsy device 217, an actuator assembly 230, and an ejection device 232. The biopsy device 217 can include both the collection member 218 and the cutting member 222 (FIG. 12). The actuator assembly 230 can include a first portion 264 that actuates the collection member 218, a second portion 266 that actuates the cutting member 222, and a third portion 268 that retracts both the collection member 218 and the second portion 266. Actuation of the third portion 268 also causes the ejection device 232 to eject the hemostatic agent 228 (FIG. 14).

The first portion 264 can include a first stage 270, a first retainer 272, and a first biasing member 274 (FIG. 11). Likewise the second portion 266 can include a second stage 288, a second retainer 294, and a second biasing member 298 (FIG. 11). The first and second portions 264, 266 share some similarities with the embodiments of FIGS. 1-10.

The third portion 268 can include a third stage 306, a third retainer 308, and a third biasing member 310 (FIG. 11). The third stage 306 can include a substantially hollow tube with a first wall 501, a second wall 503, a third wall 505, a fourth wall 507, and a fifth wall 509, which are spaced apart to divide the third stage 306 into different chambers. The third stage 306 is slidingly disposed within the housing 12 to slide along the axis X.

In the first position shown in FIG. 11, the first retainer 272 releasably engages the first wall 501, leaving the first biasing member 274 compressed between the second wall 503 and the first stage 270. Also, the second retainer 294 releasably engages the third wall 505, leaving the second biasing member 298 compressed between the third wall 505 and the second stage 288. Additionally, the third retainer 308 releasably engages first projections 511 of the housing 212 that extend through slots 513 of the third stage 306. This leaves the third biasing member 310 compressed between the end of the housing 212 and the fifth wall 509.

Moreover, the ejection device 232 includes a head member 515 that is slidingly disposed within the first stage 270 (FIG. 11). The head member 515 includes clips 517 on one end that can selectively engage second projections 519 of the housing 512. The ejection device 232 can also include a fourth biasing member 521, such as a helical compression spring, that is disposed between the head member 515 and the internal surface of the first stage 270. In addition, the ejection device 232 can include a ram rod 523 that can extend longitudinally through the cannula of the collection member 218. The ram rod 523 can be axially straight and can be relatively stiff. As will be discussed, retraction of the collection member 218 by the actuator assembly 230 can cause the ram rod 523 to push the hemostatic agent 228 out of the collection member 218.

Assuming the assembly 210 is in the position shown in FIG. 11, the user begins operation by depressing the button 84. This causes the first retainer 272 to release similar to the embodiments of FIGS. 1-10, and the first stage 270 and head member 515 move as a unit along the axis X due to the biasing force supplied by the first biasing member 274 (FIG. 12). This movement of the first stage 270 moves the collection member 218 to its extended position. This movement also causes the clips 517 of the head 515 to engage the second projections 519 of the housing 212.

Subsequently, the first stage 270 releases the second retainer 294 similar to the embodiments of FIGS. 1-10, and the second biasing member 298 biases the second stage 288 along the axis X to move the cutting member 222 to its extended position (FIG. 13).

Eventually, the second stage 288 releases the third retainer 308 from the first projections 511, thereby allowing the third biasing member 310 to bias the third stage 306 along the axis X relative to the housing 212 (FIG. 14). This movement of the third stage 306 also pushes the first and second stages 270, 288 to retract both the collection member 218 and the cutting member 222.

Because the clips 517 of the head 515 have previously engaged the projections 519, the head 515 and the ram rod 523 remains in a fixed position relative to the housing 212 as the collection member 218 retracts. Accordingly, the ram rod 523 ejects the hemostatic agent 228 from the collection member 218 as the collection member 218 retracts.

It will be appreciated that the biopsy device assembly 10, 210 can be modified in various ways. For instance, in some embodiments, the assembly 10, 210 can include a plurality of independent biopsy devices 17, 217 that are each independently actuated by the actuator assembly 30, 230. The control 82, 282 can be configured to allow the user to select which of the biopsy devices 17, 217 to actuate at selected times. Also, the actuator assembly 30, 230 (e.g., via a ratcheting system) to sequentially move each of these biopsy devices 17, 217 into and out of engagement with the first and second portions 64, 264, 66, 268 for sequentially performing the biopsies.

Moreover, the assembly 10, 210 can be configured such that the biopsy device 17, 217 is detachably connected to the other portions of the assembly 10, 210. For instance, the user may wish to detach the biopsy device 17, 217 from the assembly 10, 210 after an initial biopsy procedure and may wish to attach a fresh biopsy device 17, 217 to the assembly 10, 210 to perform a subsequent biopsy procedure.

Still further, the assembly 10, 210 may be configured to allow the user to load and reload the hemostatic agent 28, 228. In some embodiments, the biopsy device 17, 217 may be manufactured and marketed with the hemostatic agent 28, 228 preloaded therein, and after an initial biopsy procedure, the user can reload a fresh hemostatic agent 28, 228 within the same biopsy device 17, 217.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath”, “below”, “lower”, “above”, “upper” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

What is claimed is:

1. A biopsy device assembly that performs a biopsy of an anatomical tissue of a patient comprising:
a housing;
a biopsy device that extends out of the housing and that collects and cuts anatomical tissue from the patient;
a hemostatic agent removably housed in the biopsy device;
an actuator assembly that moves the biopsy device relative to the housing from a first position to an extended position such that the biopsy device collects and cuts the anatomical tissue from the patient, the actuator assembly also retracting the biopsy device relative to the housing toward a retracted position; and
an ejection device that ejects the hemostatic agent from the biopsy device as the actuator assembly retracts the biopsy device toward the retracted position.

2. The biopsy device assembly of claim 1, wherein the ejection device includes a tube with a cavity and a plunger moveably received in the cavity, wherein the biopsy device includes a cannula that is in fluid communication with the cavity, the plunger moving within the cavity when the actuator assembly retracts the biopsy device to increase pressure in the cavity and the cannula to eject the hemostatic agent.

3. The biopsy device assembly of claim 2, wherein the biopsy device includes a collection member with a collection recess and a cutting member, the actuator assembly extending the collection member out of the cutting member in a respective extended position such that the anatomical tissue is received in the collection recess, the actuator assembly moving the cutting member over the collection recess in a respective extended position to cut the anatomical tissue and to retain the anatomical tissue within the collection recess, wherein the actuator assembly includes a first stage, a second stage, a third stage, a third retainer, and a third biasing member, the first stage operably coupled to the collection member, the second stage operably coupled to the cutting member, the third stage operably coupled to the plunger, the third retainer releasably retaining the third stage in a first position, the second stage selectively releasing the third retainer, the third biasing member biasing the third stage to move the plunger within the cavity to increase pressure in the cavity and the cannula to eject the hemostatic agent.

4. The biopsy device assembly of claim 1, wherein the hemostatic agent is housed in a cannula in the biopsy device, and wherein the ejection device includes a ram rod that extends through the cannula, the ram rod pushing the hemostatic agent out of the cannula as the actuator assembly retracts the biopsy device toward the retracted position.

5. The biopsy device assembly of claim 4, wherein the biopsy device includes a collection member with a collection recess and the cannula, the biopsy device also including a cutting member, the actuator assembly extending the collection member out of the cutting member in a respective extended position such that the anatomical tissue is received in the collection recess, the actuator assembly moving the cutting member over the collection recess in a respective extended position to cut the anatomical tissue and to retain the anatomical tissue within the collection recess, wherein the ram rod includes a head that selectively engages the housing in a substantially fixed position as the collection member moves toward the respective extended position, the actuator assembly including a third stage that pushes the collection member and the cutting member toward respective retracted positions thereby causing the ram rod to push the hemostatic agent out of the cannula.

6. The biopsy device assembly of claim 1, wherein the biopsy device includes a collection member with a collection recess, the biopsy device also including a cutting member, the actuator assembly extending the collection member out of the cutting member in a respective extended position such that the anatomical tissue is received in the collection recess, the actuator assembly moving the cutting member over the collection recess in a respective extended position to cut the anatomical tissue and to retain the anatomical tissue within the collection recess.

7. The biopsy device assembly of claim 6, wherein the collection member includes a cannula, and wherein the hemostatic agent is removably housed within the cannula of the collection member.

8. The biopsy device assembly of claim 7, wherein the collection member includes a first terminal end and a second terminal end, and wherein the cannula extends continuously between the first terminal end and the second terminal end.

9. The biopsy device assembly of claim 6, wherein the actuator assembly includes a first stage, a first retainer, and a first biasing member, the first stage operably coupled to the collection member, the first retainer releasably retaining the first stage and the collection member in a respective first position, wherein the first biasing member biases the first stage when the first retainer releases to move the collection member toward the respective extended position.

10. The biopsy device assembly of claim 9, wherein the actuator assembly includes a second stage, a second retainer, and a second biasing member, the second stage operably coupled to the cutting member, the second retainer releasably retaining the second stage and the cutting member in a respective first position, wherein the second biasing member biases the second stage when the second retainer releases to move the cutting member toward the respective extended position.

11. The biopsy device assembly of claim 10, wherein the actuator assembly further includes a third stage, a third retainer, and a third biasing member, the third stage operably coupled to the first and second stages, the third retainer releasably retaining the third stage in a respective first position, wherein the third biasing member biases the third stage when the third retainer releases to retract the first stage, the collection member, the second stage, and the cutting member.

12. The biopsy device assembly of claim 11, further comprising a control that selectively releases the first retainer, the first stage releasing the second retainer after the collection member is in the respective extended position, the second stage releasing the third retainer after the cutting member is in the respective extended position.

13. The biopsy device assembly of claim 1, wherein the actuator assembly includes an outer cylinder and an inner cylinder, the inner cylinder being received in the outer cylinder, the outer cylinder fixed to the biopsy device, the inner cylinder engaging the outer cylinder as the biopsy device is moved from the first position to the extended position, the inner cylinder moving relative to the outer cylinder to disengage from the outer cylinder as the biopsy device is moved from the first position to the extended position.

14. The biopsy device assembly of claim 13, wherein the outer cylinder includes a track, wherein the inner cylinder includes a protrusion that is moveably received in the track, and wherein the housing includes a cam member that cams the protrusion within the track to rotate the inner cylinder relative to the outer cylinder and to disengage the inner cylinder from the outer cylinder as the biopsy device moves from the first position to the extended position.

15. The biopsy device assembly of claim 1, wherein the hemostatic agent includes a hemostatic foam and a biocompatible stiffener, the hemostatic foam being fixed to the biocompatible stiffener.

16. The biopsy device assembly of claim 15, wherein the biocompatible stiffener includes an elongate backbone and a plurality of ribs that are spaced apart from each other and that are each coupled to the elongate backbone, wherein the hemostatic foam is disposed between the plurality of ribs.

17. The biopsy device assembly of claim 1, further comprising a handle member operable for selectively moving the biopsy device relative to the housing from the retracted position to the first position.

18. A method of performing a biopsy comprising:
locating a biopsy device relative to a target location within a patient, the biopsy device extending from a housing;
actuating the biopsy device relative to the housing from a first position to an extended position such that the biopsy device collects and cuts an anatomical tissue from the patient;
retracting the biopsy device relative to the housing toward a retracted position; and
ejecting a hemostatic agent from the biopsy device as the biopsy device is retracted toward the retracted position.

19. The method of claim 18, wherein ejecting the hemostatic agent comprises moving a plunger inside of a cavity to increase pressure in a cannula of the biopsy device to thereby eject the hemostatic agent out of the cannula.

20. The method of claim 18, wherein ejecting the hemostatic agent comprises abutting and pushing the hemostatic agent with a ram rod from a cannula of the biopsy device.
